(19) United States
(12) Patent Application Publication
(10) Pub. No.: US 2020/0138592 A1
(43) Pub. Date: May 7, 2020

(54) INTERLOCKING INTERVERTEBRAL SPACER AND METHOD

(71) Applicant: Peter L. Bono, Bingham Farms, MI (US)

(72) Inventors: Peter L. Bono, Bingham Farms, MI (US); James D. Lark, West Bloomfield, MI (US); John S. Scales, Ann Arbor, MI (US)

(21) Appl. No.: 16/675,714

(22) Filed: Nov. 6, 2019

Related U.S. Application Data
(60) Provisional application No. 62/756,364, filed on Nov. 6, 2018.

(51) Int. Cl. A61F 2/44

(52) U.S. Cl. CPC A61F 2/447; A61F 2002/30604; A61F 2002/4415

(57) ABSTRACT

A surgical implant particularly for use in spinal surgery. The implant has a pair of integral locking structures extending from opposite sides thereof for receipt in channels machined into orthopedic components like adjacent vertebral bodies. The implant has a main body containing channel portions forming communication between the adjacent bones and a space between the adjacent bones permitting bone growth into the space through the implant.

### PRIORITY CLAIM

In accordance with 37 C.F.R. 1.76, a claim of priority is included in an Application Data Sheet filed concurrently herewith. Accordingly, the present invention claims priority to U.S. Provisional Patent Application No. 62/756,364, entitled “INTERLOCKING INTERVERTEBRAL SPACER AND METHOD”, filed Nov. 6, 2018. The contents of the above referenced application is incorporated herein by reference in its entirety.

### FIELD OF THE INVENTION

An orthopedic implant particularly adapted for use in spinal surgery.

### BACKGROUND OF THE INVENTION

Surgical procedures, such as those performed on the spine, are well known in the art. The central nervous system is a vital part of the human physiology that coordinates human activity. It is primarily made up of the brain and the spinal cord. The spinal cord is made up of a bundle of nerve tissue which originates in the brain and branches out to various parts of the body, acting as a conduit to communicate neuronal signals from the brain to the rest of the body, including motor control and sensations. Protecting the spinal cord is the spinal, or vertebral, column, Anatomically, the spinal column is made up of several regions, including the cervical, thoracic, lumbar and sacral regions. Each of the vertebrae associated with the various spinal cord regions are made up of a vertebral body, a posterior arch, and a transverse process.

While most people have fully functional spinal cords, it is not uncommon for individuals to suffer some type of spinal ailment or disorder that can be treated by some type of surgical intervention. There are many different approaches taken to alleviate or minimize severe spinal disorders. One surgical procedure commonly used is a spinal fusion technique. Several surgical approaches have been developed over the years, and include the Posterior Lumbar Interbody Fusion (PLIF) procedure which utilizes a posterior approach to access the patient's vertebrae or disc space; the Transforaminal Lumbar Interbody Fusion (TLIF) procedure which utilizes a posterior and lateral approach to access the patient's vertebrae or disc space; and the Anterior Lumbar Interbody Fusion (ALIF) which utilizes an anterior approach to access the patient's vertebrae or disc space. Using any of these surgical procedures, the patient undergoes spinal fusion surgery in which two or more vertebrae are linked or fused together through the use of a bone spacing device, screws, rods and/or the use of bone grafts. The resulting surgery eliminates any movement between the spinal sections which have been fused together.

In addition to the spinal implants or use of bone grafts, spinal fusion surgery often utilizes spinal instrumentation or surgical hardware, such as pedicle screws, cages, plates, or spinal rods. Once the spinal spacers and/or bone grafts have been inserted, a surgeon places the pedicle screws into a portion of the spinal vertebrae and attaches either rods or alternatively plates with screws as a means for stabilization while the bones fuse. Currently available systems for inserting the rods into pedicle screws can be difficult to use, particularly in light of the fact that surgeons installing these rods often work in narrow surgical sites.

Moreover, since patients can vary with respect to their internal anatomy, resulting in varying curvatures of the spine, a surgeon may not always have a linear path, or may have anatomical structures that must be maneuvered around in order to properly insert the surgical rods into the pedicle screw assemblies. In addition to requiring surgical skill, difficulty in placing the rods correctly into the pedicle screws can result in unnecessary increases in the time it takes a surgeon to complete the surgical procedure. Prolonged surgery times increase the risk to the patient. More importantly, improperly aligning the rods and pedicle screw assemblies sometimes results in post-surgery complications for the patient and requires corrective surgical procedures.

Robotic surgery, computer-assisted surgery, and robotically-assisted surgery are terms for technological developments that use robotic systems to aid in surgical procedures. Robotically-assisted surgery was developed to overcome the limitations of pre-existing minimally-invasive surgical procedures and to enhance the capabilities of surgeons performing open surgery.

In the case of robotically-assisted minimally-invasive surgery, instead of directly moving the instruments, the surgeon uses one of two methods to control the instruments; either a direct telemanipulator or through computer control. A telemanipulator is a remote manipulator that allows the surgeon to perform the normal movements associated with the surgery while the robotic arms carry out those movements using end-effectors and manipulators to perform the actual surgery on the patient. In computer-controlled systems, the surgeon uses a computer to control the robotic arms and the end-effectors, though these systems can also still use telemanipulators for their input. One advantage of using the computerized method is that the surgeon does not have to be present, but can be anywhere in the world, leading to the possibility for remote surgery. One drawback relates to the lack of tactile feedback to the surgeon. Another drawback relates to visualization of the surgical site. Because the surgeon may be remote or the surgery may be percutaneous, it is difficult for the surgeon to view the surgery as precisely as may be needed.

While many devices and methods are available for use in spinal surgery, they typically have limits on their use. Thus, there is still a need for new devices that improve on the current devices and methods.

### DESCRIPTION OF THE PRIOR ART

PEEK (poly-ether-ether-ketone) spacers are used both in the posterior vertebral arch and inter-body to reposition one vertebra relative to an adjacent vertebra. Devices and processes are available to provide disc replacement if the disc needs replaced. Spinal rods attached to the spine with pedicle screws have also been used to correct spinal problems. Bone spacers attached in place with metal plates are secured in place with bone screws attached to vertebral bodies. Bone grafts are also used to fuse the bone around the implant. Examples of such devices can be found disclosed in U.S. Patent Application Publications 2018/0289502, 2018/0289501, 2018/0289496, 2018/0290154, 2018/0250142 and 2018/0200063. These applications illustrate such devices and their differences, showing the amount of effort being devoted to making improved devices.

### SUMMARY OF THE INVENTION

It is an object of the present invention to provide an improved orthopedic implant particularly configured to be used as an implant for attachment to two adjacent vertebrae. The implant interlocks with two adjacent vertebrae to provide spacing between the vertebrae while preventing the vertebrae from moving with respect to each other. The implant is constructed allow for bone ingrowth in and through the implant to provide for stronger fusions.

Accordingly, it is a primary objective of the present invention to provide an implant that can be used to retain one vertebra positioned relative to an adjacent vertebra.

It is a further objective of the present invention to provide an implant that can be attached to two adjacent vertebrae bodies and retain them in a desired position relative to one another.

It is another objective of the present invention to provide an implant and method that can use current surgical methods and equipment to effect attachment of the implant to a pair of adjacent vertebrae.

It is yet another objective of the present invention to provide a spinal implant that interlocks with adjacent vertebrae to maintain positioning.

It is still yet another objective of the present invention to provide a spinal implant that cooperates with a T-slot, dovetail or the like cut into each adjacent vertebra.

Other objects and advantages of this invention will become apparent from the following description taken in conjunction with any accompanying drawings wherein are set forth, by way of illustration and example, certain embodiments of this invention. Any drawings contained herein constitute a part of this specification, include exemplary embodiments of the present invention, and illustrate various objects and features thereof.

### BRIEF DESCRIPTION OF THE FIGURES

FIG. 1 is a side elevation view of an implant attached to a pair of adjacent vertebrae;

FIG. 2 is an isometric view of one form of the orthopedic implant shown in FIG. 1;

FIG. 3 is an isometric view of a second form of the orthopedic implant shown in FIG. 1;

FIG. 4 is a side elevation view of a third form of the orthopedic implant; and

FIG. 5 is an isometric view of the implant of FIG. 4.

### DETAILED DESCRIPTION OF THE INVENTION

Curved spine conditions such as Scoliosis, Kyphosis and Lordosis may need surgical intervention to correct and provide comfort and functionality for a patient. Scoliosis can be congenital and can be treated with surgical intervention. Kyphosis can be congenital from developmental abnormalities and can occur in adolescence from asymmetric spine development or infection. Lordosis can also be congenital and can be caused by trauma. Curved spine conditions can be treated surgically with orthopedic implants.

Failed discs may likewise need surgical intervention. Such intervention is typically surgical using some implant device to retain one vertebra in a desired position relative to an adjacent vertebra.

The present invention provides an implant usable in orthopedic procedures. Such orthopedic procedures preferably utilize a surgical robot to perform at least a portion of the procedure. Such a robot includes an effector used to remove tissue, such as bone. The implant of this invention has a pair of integral locking structures extending from opposite sides of a central body for receipt in channels machined into orthopedic components such as adjacent vertebral bodies. The implant has a central or main body containing channel portions, forming communication between the adjacent bones and a space between the adjacent bones, permitting bone growth into the space through the implant.

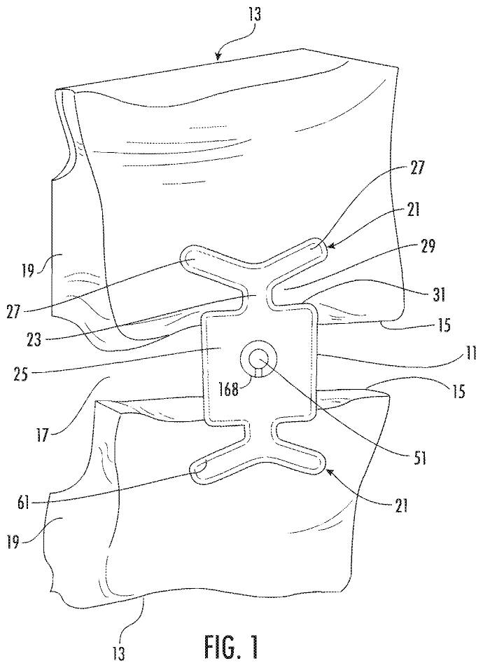

FIG. 1 shows an implant 11 secured to and extending between a pair of adjacent vertebral bodies 13. The vertebral bodies 13 have adjacent and opposing faces 15 between which disc material (not shown) is normally contained. As shown, there is a space 17 between the faces 15 in which the disc material is normally positioned. On the posterior side of the vertebral bodies 13 there is the posterior vertebral arch which includes pedicles 19. The structure and functionality of a vertebra and other components of the spine are well known in the art. Vertebral bodies and the posterior vertebral arch protect the spinal cord.

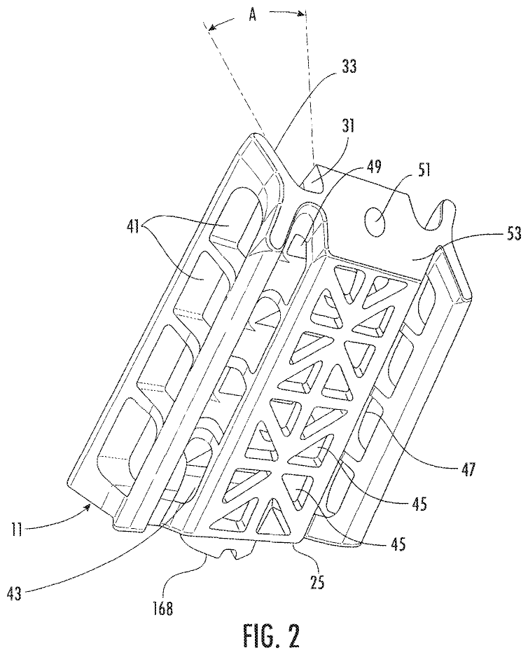

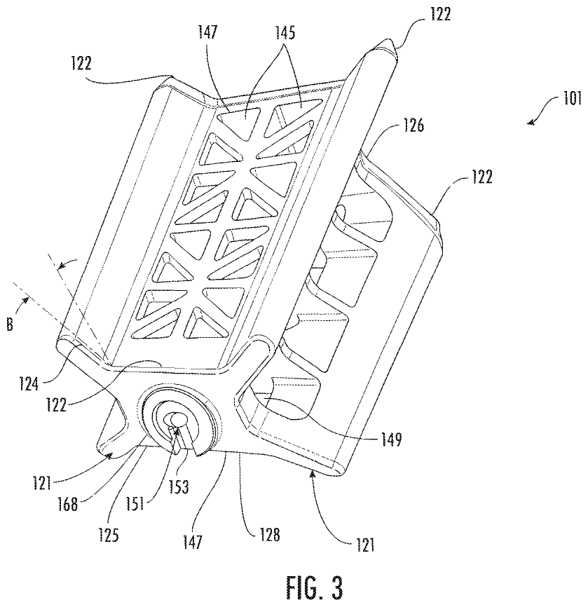

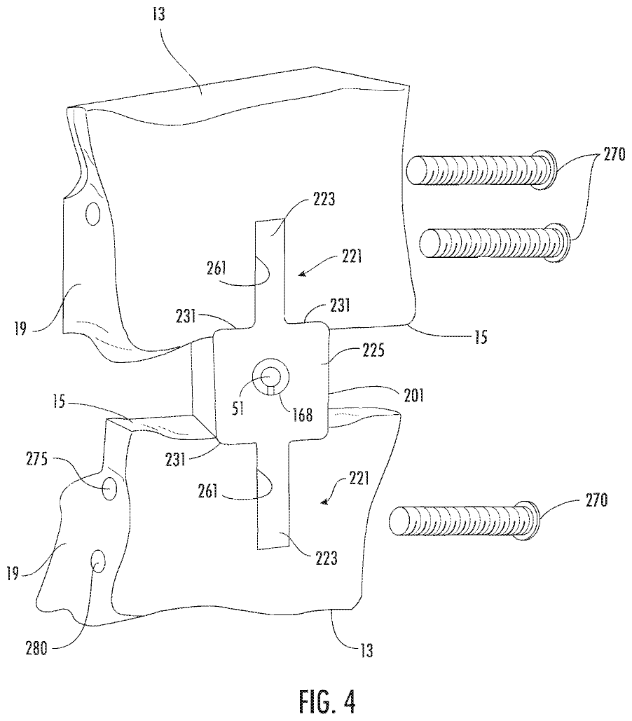

FIG. 2 illustrates the implant 11 as seen in FIG. 1. The implant 11 (and others described herein) can be made of a metallic or polymeric material. A suitable metal material can be titanium-based, such as titanium or titanium alloy, which hereinafter will be referred to as titanium and may include printed porous or foamed titanium. A suitable polymeric material can be poly-ether-ether-ketone (PEEK). The United States FDA approves materials for use in contact with animal tissue, including human tissue, as do other similar government agencies. Both titanium and PEEK have been approved by the FDA. A metallic implant 11 can be made by 3D printing, which forms an implant that is porous as opposed to solid metal, providing voids for helping attach the implant 11 to new bone growth. The implants shown in both FIGS. 2, 3 are configured to mechanically lock in place through their own structure, while the implant shown in FIG. 4 can be held in place mechanically with fasteners, such as screws as described below.

The implant 11 has a pair of integral locking structures, designated generally 21, and can be similar or identical in configuration and size, though they need not be. The locking structures extend from opposite sides of a main body portion 25. As shown, the locking structures 21 include a web 23 extending from the main body portion 25 to connect the body 25 to one or more locking flanges 27. As illustrated, the web 23 and flanges 27 form a Y shape in transverse cross-section. The flanges 27 form a V shape. The locking structures 21 form a mechanical interlock with the bone in which it is installed. While a pair of flanges 27 are shown, it is to be understood that a single flange 27 could be used so long as there is at least one undercut 29 formed to lock the implant 11 to a respective vertebral body 13. While a Y shape is shown, it is to be understood that the locking structures 21 can be T-shaped, L-shaped, dovetail shaped or may include any other shape in transverse cross section suitable for interlocking with the adjacent vertebrae. Preferably, the angle between a shoulder 31 that engages a face 15 of the vertebral body 13 and a surface 33 facing the shoulder 31 is between about +45° to about −15°. Preferably, the shoulder 31 would be parallel to a respective face 15 when the implant 11 is installed. The angle A as shown in FIG. 2 is a positive angle. If the surface 33 and shoulder 31 are parallel for a T-shaped locking structure 21, the angle A would be 0°.

The implant 11 has a plurality of interconnecting channels for the insertion of bone graft material, such as cadaver bone, into the interior of the implant 11 to assist in bone growth between the vertebral bodies 13 in the space 17 after the implant 11 is installed. The flanges 27 have channel portions 41 therethrough. As shown, the web 23 is also provided with channel portions 43 therethrough the main body 25 has a channel portions 45 through opposite side panels 47 that open into channel portions 49 between the opposite side panels 47. One or more through openings 51 can be provided in the end panel 53 to allow the surgeon to pack the implant 11 with bone material after it is installed to help subsequently better secure the implant 11 in place. The main body 25 and locking structures 21 form scaffolds to assist in bone growth. The main body 25 has the channel portions 41, 43, 45 forming communication between the adjacent bones 13 and the space 17 between the adjacent bones, permitting bone growth into the space through the implant. The locking structures 21 mechanically lock the implant 11 through interference with part of the respective vertebral body 13 as described below.

In use, a surgeon will use a robot such as that disclosed in our co-pending Patent Application Nos. 62/616,673, 62/681,462, 62/423,651, 62/616,700 and Ser. No. 15/816,861 to Peter L. Bono. These applications disclose robotic surgical systems usable with the present system. The entireties of these disclosures are incorporated herein by reference. Such systems can be used to effect removal of tissue from the vertebral bodies 13 to form channels 61 for receipt of the locking structures 21, 121 (structure 121 is described below). The locking structures 121 each include a pair of integral flanges 122 extending from the top 126 and bottom 128 of a main body portion 125 as more fully described below. The top 126 and bottom 128 are in essence the same in the illustrated embodiment. As shown, a channel 61, is similar both in length and transverse cross-sectional shape to that of a respective locking structure 21. The channels 61 can be formed using a robot with an appropriate effector (not shown). Once the channels 61 are machined into the vertebral bodies 13, the implant 11 is installed by the application of longitudinally directed force on the implant 11, as with a hammer or other force applying device such as the surgical robot. The surgeon can dress the faces 15 prior to inserting the implant 11, but need not remove all the disc material. Removal of disc material is problematic; and the present invention reduces or eliminates the need to remove all the disc material except the disc material that would engage the shoulders 31. After installation of the implant 11, the patient takes care to allow the bone material to grow at the site of the implant 11, and ultimately throughout the space 17 between the faces 15. There is, thus, with this embodiment, no need for separate mechanical fasteners to secure the implant 11 in place and to retain it in place during the healing period.

FIG. 3 illustrates a second embodiment of an implant. This implant embodiment is designated generally 101. The main difference between the implant 11 and the implant 101 is the elimination of the web 23. The implant 101 includes the main body portion 125 having a series of channel portions 145 through opposite side panels 147 that open into channel portions 149 positioned between the opposite panels 147, as described above for the main body portion 25 channel portions 43 opposite side panels 47 and channel portions 45. One or more through openings 151 can be provided in the end the panel 153 to allow the surgeon to pack the implant 101 after it is installed with bone material to help subsequently better secure the implant 101 in place after it is installed. The main body 125 and locking structures 121 form scaffolds to assist in bone growth, as described above for the implant 11 shown in FIG. 3

The implant 101 is provided with locking structures designated generally 121. As shown, the locking structures 121 are Y-shaped, having an angle B between a line perpendicular to the side edge in the face 124 of between about 20° and about 70°. While the term “about” is used, that means that angles A, B are approximations given normal manufacturing tolerances and the ability to measure these angles and variations in the reference surfaces from manufacturing.

As shown in the implant 101 of FIG. 4, the implant is provided with means for the surgical robot to pick up, hold and orient the implant 101 for insertion into the milled channels 61. This is illustrated as a coupler device 168. The use and installation of the implant 101 is similar to that for the implant 11 described above.

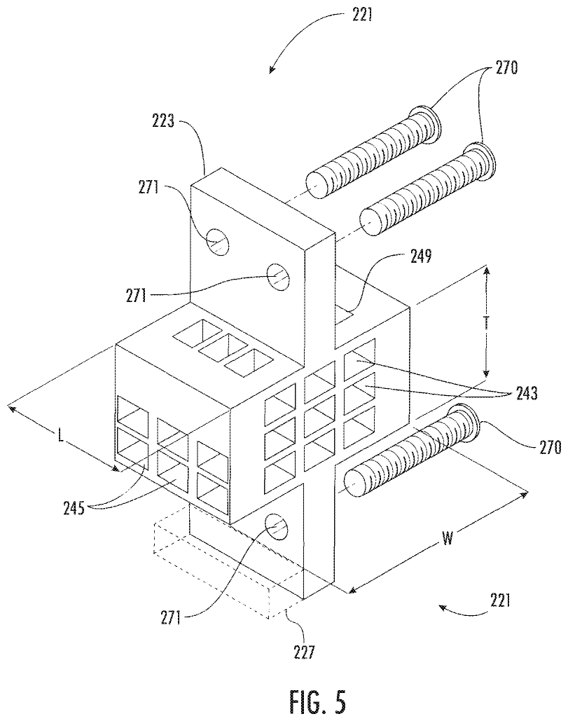

FIGS. 4 and 5 illustrate a third embodiment of the present invention. It shows an implant 201 constructed and configured to fit between two adjacent vertebral bodies 13 similar to the implants 11 and 101. The implant 201 has a main body 225 that can be constructed with channel portions 243, 245, 249 similar to the channel portions 41, 43 and 45. The channel portions 243, 245, 249 serve the same function as the channel portions 41, 43, 45 and can be similarly constructed. The main body can have a shoulder 231, like shoulder 31, to engage a respective portion of a face 15. The implant 201 can be provided with a coupler device 168 and one or more through openings, like the opening 51 shown in FIG. 2. As shown, the implant 201 is provided with a pair of oppositely extending integral flanges 223, which serves as a portion of a locking structure 221 to help secure the implant 201 between two adjacent vertebral bodies 13. While the flanges 223 are shown as generally planar, it is to be understood that they can be provided with generally laterally projecting side flanges 227, as shown in dashed lines in FIG. 5 on one flange 223. Each of the flanges 223 is received within a respective vertebral body channel 261 to assist in locking the implant 201 to the vertebral bodies 13. The locking structure 221 in the illustrated embodiment includes a mechanical fastening device, or fastener, 270 for each flange 223 to mechanically secure the implant 201 in position between the two vertebral bodies 13 such that the shoulders 231 engage respective faces 15. Preferably, two fasteners 270 are used for each flange 223. As seen in FIG. 4, the fasteners 270 that are secured to the respective vertebral body 13 and through a respective aperture 271 are bone screws. The fasteners 270 can extend initially through a pre-drilled hole 275 then pass through a respective aperture 271, and then threadably engage the respective vertebral body on the downstream side of the respective aperture 271. The fasteners 270 then mechanically secure the implant 201 to the vertebral bodies 13.

As mentioned above, side flanges 227 can be provided on either or both of the flanges 223 to be received in a portion of the channel 261 (not shown) that extends laterally from the portion that the flange 223 is received in, similar to the construction of the implants 11 and 101. It is also to be noted that, if rods are to be used, as is known in the art to help align the vertebrae, pedicle screws can be used in place of the fasteners 270 and would be installed through, for example, a hole 280 in a pedicle portion 19 of the vertebra, as is known in the art. The surgical robot can identify the location of the implant 201 and, through programming, know where the apertures 271 are and direct the attachment of the fasteners 270 so they pass through a respective aperture 271.

While the implant embodiments 11, 101, 201 described above are substantially rigid, it is to be understood that, in some applications not involving a spine, they could be flexible to bend with a joint that needs to flex. They can also have the shoulder 231 surface pre-contoured to match the touching portion of face 15. Implants can be provided that have different lengths (L), widths (W) and thicknesses (T) to accommodate different patients. A preferred installation of an implant would be laterally across the spine, but anterior installation and even an angular installation, say more from the posterior while avoiding the spinal cord, could be done particularly with the implants 11 and 101 that do not use fasteners.

All patents and publications mentioned in this specification are indicative of the levels of those skilled in the art to which the invention pertains.

It is to be understood that while a certain form of the invention is illustrated, it is not to be limited to the specific form or arrangement herein described and shown. It will be apparent to those skilled in the art that various changes may be made without departing from the scope of the invention, and the invention is not to be considered limited to what is shown and described in the specification and any drawings/figures included herein.

One skilled in the art will readily appreciate that the present invention is well adapted to carry out the objectives and obtain the ends and advantages mentioned, as well as those inherent therein. The embodiments, methods, procedures and techniques described herein are presently representative of the preferred embodiments, are intended to be exemplary, and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

What is claimed is:

1. A surgical implant for bridging between two adjacent bones each having an attachment second channel formed therein, the implant comprising;
a main body containing first channel structure forming communications between adjacent bones in a space between the bones when the implant is installed in a patient said main body having a pair of opposite facing shoulders adapted to engage adjacent bones when the implant is installed; and
a pair of locking structures each extending from a respective said shoulder adapted to be received in a second channel formed in a respective bone.

2. The implant as set forth in claim 1 wherein at least one of said locking structures having a first portion shaped to form a mechanical interlock with a bone in which it is installed.

3. The implant as set forth in claim 2 wherein at least one of said locking structures being generally Y shaped.

4. The implant of claim 2 wherein said pair of locking structures both being shaped to form a mechanical interlock with a bone in which it is installed.

5. The implant of claim 1 wherein said first channel structure having portions opening onto said oppositely facing shoulders.

6. The implant of claim 5 wherein said first channel structure having portions opening onto portions of said locking structures.

7. The implant of claim 6 wherein said main body having side panels each extending between a pair of respective said shoulders, said first channel structure having portions opening onto said side portions.

8. The implant of claim 1 wherein at least one said locking structure including a flange extending from a respective pair of said shoulders for receipt in a respective said second channel.

9. The implant of claim 1 including a coupler device associated with said main body.

10. An orthopedic surgical method including:
forming second channels in adjacent bones in a patient;
placing an implant having a main body containing a first channel structure forming bone growth communication between adjacent bones in a space between the bones, said main body having a pair of opposite facing shoulders engaging said adjacent bones; and
positioning a pair of locking structures of said implant each into a respective said second channel.

11. The method as set forth in claim 10 wherein at least one of said locking structures having a first portion forming a mechanical interlock with the bone in which it is installed.

12. The method as set forth in claim 11 wherein said pair of locking structures each having a first portion forming a mechanical interlock with the bone in which it is installed.

13. The method as set forth in claim 10 wherein the adjacent bones have a space therebetween and said first channel structure having portions forming communication with said space.

14. The method as set forth in claim 10 including inserting bone graft material in said first channel structure.
